(19) United States
(12) Patent Application Publication
(10) Pub. No.: US 2021/0310074 A1
(43) Pub. Date: Oct. 7, 2021

(54) PROGNOSTIC AND PREDICTIVE TRANSCRIPTOMIC SIGNATURES FOR UTERINE SEROUS CARCINOMAS

(71) Applicant: Augusta University Research Institute, Inc., Augusta, GA (US)

(72) Inventors: Jin-Xiong She, Augusta, GA (US); Lynn Tran, Augusta, GA (US)

(73) Assignee: Augusta University Research Institute, Inc., Augusta, GA (US)

(21) Appl. No.: 17/173,931

(22) Filed: Feb. 11, 2021

Related U.S. Application Data
(60) Provisional application No. 62/972,920, filed on Feb. 11, 2020.

(51) Int. Cl. C12Q 1/6886; G16B 25/10; G16B 5/00

(52) U.S. Cl. CPC C12Q 1/6886; G16B 25/10; G16B 5/00; C12Q 2600/118; C12Q 2600/112; C12Q 2600/106; C12Q 2600/158

(57) ABSTRACT

The application provides methods of prognosing and classifying uterine serous carcinoma (USC) patients into poor survival groups or good survival groups and for predicting response to therapy by way of a multigene signature. The application also includes kits and computer products for use in the methods of the application.

### CROSS-REFERENCE TO RELATED APPLICATIONS

This application claims benefit of and priority to U.S. Provisional Application No. 62/972,920 filed on Feb. 11, 2020, which is incorporated by reference in its entirety.

### FIELD OF THE INVENTION

The invention relates to transcriptomic biomarkers associated with uterine serous carcinomas (USC), methods for the prognosis of USC and for predicting patient response to therapy.

### BACKGROUND OF THE INVENTION

Endometrial cancer is the most common gynecologic malignancy and the 4^{th} most common overall malignancy in women, with over 61,000 estimated new cases in the US in 2019 and over 11,000 deaths. Over half of these deaths are attributed to an uncommon subtype, uterine serous carcinoma (USC), which only represents 10% of new endometrial cancer cases. The standard of care for these patients consists of surgical resection followed by carboplatin and paclitaxel chemotherapy with or without radiation. However, the 5-year overall survival (OS) rate is only ˜50%, which is partly attributable to the presence of distant metastases in 60% of patients. From the OS and metastasis rates, it is apparent that there is significant heterogeneity in the clinical outcome. To better tailor treatments, accurate predictors of patient survival and response to standard therapy are needed. To this end, multiple studies attempted to develop molecular markers to aid in prognostication of, and therapeutic selection for, USC. Genetic studies have revealed that USC, in contrast to other subtypes, often has mutations in p53, PIK3CA, PIK3R1, HER2/Neu and PTEN. Mutation-targeted trials, however, have failed to yield a survival advantage as monotherapy, suggesting that these mutations are insufficient therapeutic biomarkers. Many molecular markers have also been identified as potential prognostic, recurrence, and/or therapeutic biomarkers, including CA125, HER2, hormone receptors such as ER and PR, cellular proliferation proteins, and DNA ploidy/copy number, amongst others. None of these biomarkers has been implemented clinically. Instead, patient prognosis is often evaluated using clinical and demographic variables.

Despite an abundance of potential predictive markers, none of these markers can clearly resolve how long patients will survive with standard therapy. All USC patients are now presumed to have poor prognosis, when in reality, this subtype is very heterogeneous, with half of the patients surviving a median of 2.5 years and half surviving well beyond 5 years. This illustrates the need to further subdivide these patients in order to identify poor prognostic patients who require different treatments. Thus, there exists a demand for a method which predicts USC survival and response to treatment.

### SUMMARY OF THE INVENTION

Transcriptomic signatures that function as very sensitive prognostic biomarker for USC were identified. The biomarkers predicted the overall survival (OS) of USC patients. In addition, the transcriptomic signatures serve as therapeutic biomarkers to guide patient care. The transcriptomic biomarkers provide prognostic indicator of uterine serous carcinoma survival and response to treatment.

Transcriptomic biomarkers were identified to distinguish or differentially prognosticate between USC patients with good versus poor survival prognosis. The transcriptomic biomarkers comprised molecules some of which were up-regulated, down-regulated, no change, absent, etc. (i.e., differentially expressed) as compared to normal healthy controls. The transcriptomic biomarkers not only allow for the prognosis and prognostic differentiation between early and late stage USC, but also for identifying a USC patient's response to treatment.

One embodiment provides a method for predicting the outcome of a subject's overall survival (OS) for uterine serous carcinoma (USC) by obtaining gene expression levels from a tumor sample from the subject of the genes selected from the group consisting of CNOT1, C1orf106, ACRC, MEIS3, HGS, GALNTL2, C8orf4, GALNTL4, IBTK, WNT7B, PHLDA2, DENND2A, C1orf126, IER3, FLJ35776, MYEOV, BTBD16, S100A10, MC1R, GNAL, RBMS2, MST1R, IL1R2, KCNE4, COL18A1, CUBN, CHRNA10, TAL1, S100A6, MMP10, S100A11, GPR124, EIF2B2, WDR17, OBFC2A, HABP2, C10orf47, GRIA3, LOC728264, COL4A4, ATG16L2, TXK, C17orf70, GPR111, COL1A1, HS3ST2, RHOV, SLC6A13, DOK4, DKK1, FLJ23867, PADI1, LIPG, LY6H, ZNF69, C2CD4A, C11orf41, VIL1, C11orf9, AG2, ERBB2, IL6, C3orf66, OVGP1, SAA4, NCOA, NPAS2, ITGA10, SH2D3A, C12orf27, CLDN14, F3, PAPPA and subcombinations thereof; optionally normalizing the expression level to the expression of a housekeeping gene; calculating a score of the gene expression levels using elastic net regression, wherein each gene is weighted; and wherein a score of less than 9 indicates a longer OS for the subject, compared to a USC patient with a score higher than 9. In some embodiments, the housekeeping gene is selected from the group consisting of actin, GAPDH and ubiquitin.

Still another embodiment provides a method of selecting a treatment for a subject with USC by classifying subjects with USC into poor response to treatment groups or good response to treatment groups using the method described above, wherein patients with a score of less than 9 indicates the patient will have a good response to standard treatment and patients with a score above 9 will have a good response to treatment with standard treatment; and treating the patients with a score of less than 9 with standard treatment selected from the group consisting of resection, chemotherapy, radiation, or a combination thereof.

Yet another embodiment provides a method of prognosis or classification of a subject having USC, by determining the score of a subject using the method described above, wherein the stage progression of USC is early stage (I & II) if the score is below 9 or the USC is advanced stage (III & IV) if the score is above 9. When the subjects with a score higher than 9 or an advanced stage classification correlates with poor prognosis and a 5-year OS of 0% to 11.6%. When the subject's score is lower than 9, an early stage classification correlates with intermediate prognosis and a 5-year OS of 45% to 82.7%.

### BRIEF DESCRIPTION OF THE DRAWINGS

FIGS. 1A-1F show Kaplan-Meier survival curves for representative single genes. Patients in each dataset were divided into high versus low expression groups and Cox proportional hazard analysis was used to compare survival between the groups defined by each individual gene. The log-rank p-value is reported for each analysis.

FIGS. 2A-2D show Kaplan-Meier survival curves between patient groups determined by USC73 score alone or together with pathologic stage. 2A) TCGA patients with high versus low USC73 scores, 2B) AU patients with high versus low USC73 scores. 2C) TCGA patients are divided into 3 groups: USC73_low, USC73_high early stage, and USC73_high advanced stage patients. 2D) AU Patients are clustered into four groups using USC73 score and pathological stage. HR is derived by comparing the patient group with low USC73 score and early stage as reference. The overall log-rank p-value is reported for each model. For all analyses, α=0.05, and bolded p values are <0.05.

FIGS. 3A-3E show Molecular and functional characteristics in USC primary cell lines. A) Scratch assay for cell migration. B) Cell growth rate. Cell numbers viability for 96 hours in 24 hour intervals. Each data point represents individual cell lines. Each measurement is conducted in triplicate. Statistical differences were quantified by one-tailed two-sample t test (both migration and growth distributions had no signification differences in variance according to Levene's homogeneity of variance test, and no significant deviation from normality according to the Wilk-Shapiro test of normality). C) Cell cycle progression of primary USC cell lines measured by propidium iodide staining. There is a negative correlation between G1 to S/M/G2 ratio and USC73 score. D) Quantification of Ki-67 staining of AU cohort (immunostaining of tissue microarray), n=41 (USC73_high n=16; USC73_low n=25). USC73 status of patient FFPE tissue measured using NanoString custom gene expression array.

FIGS. 4A-4C show Clinical and molecular characteristics of patients in subgroups of patients defined by USC73 scores. 4A) Representative staining for Ki-67 antibody (10× magnification); 4B) Bar charts showing the percentages of different categories of objective response. Complete response rate is 89.3% for USC73_low versus 55.6% for USC73_high patients. Conversely, progressive disease is observed in 7.1% and 27.8% of patients in the USC73_low and UC73_high groups. χ² test p=0.018, α=0.05; 4C) H-score for Ki-67 staining for all TCGA patients. The mean H-score is 1.5-fold higher in USC73_high patients than USC73_low patients, p=0.056 with two sample t-test and equal variance assumption.

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, the terms “transcriptomic signature”, “gene signature”, “signature”, “biomarker”, “molecular marker”, or “transcriptomic biomarker” are used interchangeably and refer to the biomolecules identified in Table 3-5. Thus, Table 3 comprising the biomolecules listed therein, represents 73 USC gene signature genes based on gene functions and potential relevance to cancer; Table 4 comprising the biomolecules listed therein, represents the computed score, termed USC73, indicating the different weights for each gene signature; and Table 5 comprising the biomolecules listed therein, represents USC73 gene signature genes that remain individually prognostic in the validation (AU) cohort. As more biomolecules are discovered, each newly identified biomolecules can be assigned to any one or more gene or transcriptomic signature. Each biomolecule can also be removed, reassigned or reallocated to a transcriptomic signature. Any one of the signatures can be used for the prognosis and prognostic differentiation between early and late stage USC, but also for identifying a USC patient's response to treatment the prognosis of USC.

The term “biomolecule” refers to genes, DNA, RNA (including mRNA, rRNA, tRNA and tmRNA), nucleotides, nucleosides, analogs, polynucleotides, peptides and any combinations thereof.

Expression/amount of a gene, biomolecule, or biomarker in a first sample is at a level “greater than” the level in a second sample if the expression level/amount of the gene or biomarker in the first sample is at least about 1 time, 1.2 times, 1.5 times, 1.75 times, 2 times, 3 times 4 times, 5 times, 6 times, 7 times, 8 times, 9 times, 10 times, 20 times, 30 times, the expression level/amount of the gene or biomarker in the second sample or a normal sample. Expression levels/amounts can be determined based on any suitable criterion known in the art, including but not limited to mRNA, cDNA, proteins, protein fragments and/or gene copy. Expression levels/amounts can be determined qualitatively and/or quantitatively.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

The term “housekeeping gene” typically refers to constitutive genes that are required for the maintenance of basic cellular function, and are expressed in all cells of an organism under normal and patho-physiological conditions.

Exemplary housekeeping genes include, but are not limited to, actin, GAPDH and ubiquitin.

### II. Transcriptomic Signatures/Molecular Biomarker

The present invention relates to transcriptomic signatures that function as very sensitive prognostic biomarkers for USC. These biomarkers not only allow for the prognosis and prognostic differentiation between early and late stage USC, but also for identifying a USC patient's response to treatment.

Uterine serous carcinoma (USC) is a highly aggressive variant of endometrial cancer. Although it only represents less than 10% of all cases, it accounts for a disproportionate number of deaths from endometrial cancer. Studies of genes with abnormal expression in endometrial cancer have identified multiple oncogenes, tumor suppressors, mismatch repair genes, apoptosis-associated genes, levels of hormone receptors and DNA ploidy and aneuploidy as biomarkers of endometrial cancer. The use of these molecules and genes may facilitate accurate diagnosis and prognostic prediction and contribute to individualized treatment. Trials of drugs which target these biomarkers and searches for new biomarkers using cDNA microarrays and RT-qPCR are ongoing and it is likely that these findings can be translated to clinical use.

Transcriptomics have emerged as a highly valuable tool to aid in complex pathologic diagnosis and prognosis. The Cancer Genome Atlas (TCGA) program acquired different types of molecular data on the three main subtypes of uterine cancer and provides a rich data set for answering various questions. The TCGA developed a uterine cancer classification system which identified subgroups based on potential molecular drivers. However, their analyses did not address the prognosis question within the USC subtype Thus our group focused on identifying transcriptomic biomarkers to differentiate USC patients with good versus poor survival prognosis. The 73 gene signature (USC73) presented in the present invention was originally discovered using publicly available transcriptomic data from the TCGA and then validated in an independent cohort of USC patients from the Medical College of Georgia at Augusta University (AU) using NanoString single molecule counting technology. The gene signature and other clinical variables were integrated to develop and validate a comprehensive prognostic predictor that could predict patient survival and assist identification of poor survival patients for testing other treatment options.

Details of the experimental procedures are provided in the examples section which follows. Briefly, analysis of the TCGA RNAseq data identified 73 genes that individually predict prognosis for USC patients and an elastic net model with all 73 genes (USC73) distinguishes good OS group with low USC73 score and 83.3% 5-year OS from a poor OS group with high USC73 score and 13.3% 5-year OS (HR=40.1; p=3×10⁻⁸). This finding was validated in the independent AU cohort (HR=4.3; p=0.0004). The poor prognosis group with high USC73 score consists of 37.9% and 32.8% of USC patients in the TCGA and AU cohort respectively. The USC73 score and pathologic stage independently contribute to OS and together provide the best prognostic value. Early stage (I & II) patients with low USC73 score have the best prognosis (5-year OS=85.1% in the combined dataset), while advanced stage (III & IV) patients with high USC73 score have the worst prognosis (5-year OS=6.4%, HR=30.5, p=1.2×10⁻¹²). Consistent with the observed poor survival, primary USC cell lines with high USC73 had higher proliferation rate and cell cycle progression; and high USC73 patients had lower rates of complete response to standard therapy.

The USC73 transcriptomic signature and stage independently predict OS of USC patients and the best prediction is achieved using USC73 and stage. USC73 may also serve as a therapeutic biomarker to guide patient care.

The disclosed method generally involves obtaining relative gene expression data at the DNA, mRNA or protein level for each of the 73 genes and any supplemental genes from the patient, processing the data, and resulting a step of comparing the obtained information with one or more reference values. Relative expression level is expression data normalized according to techniques known to those skilled in the art. Expression data can be normalized to one or more genes whose expression is invariant, such as a “housekeeping” gene.

In one aspect, a multi-gene signature for prognosis or classification of patients with uterine serous carcinomas is provided. In some embodiments, 73 different genes are identified based on outcomes such as good or poor survival and/or relative expression data for each gene from a previous data set based on potential molecular drivers for USC. A 73-gene signature is provided that contains a reference value for each of the genes.

In one aspect, for each of the 73 genes and any supplemental genes, for each gene, the relative expression data from the patient is combined with gene-specific reference values to provide prognosis or treatment recommendations. In some embodiments, the relative expression data is subjected to an algorithm that yields the USC73 score, which is subsequently compared to control values obtained from past expression data for the patient or patient pool. In some embodiments, the control value predicts the overall survival prognosis, eg, good prognosis and poor prognosis.

In one embodiment, the composite score is calculated by combining gene expression values into a linear predictor value for each patient using elastic net regression performed on TCGA gene expression data. The computed score, termed USC73, uses different weights for each gene as reported in Table 4.

The inventors have identified a multi-gene signature that is prognostic with respect to survival and predictive of gain from adjuvant chemotherapy.

Accordingly, in one embodiment, the application is a method of prognosis or classification of a subject having USC, comprising the following steps: a. Determining the expression levels of 73 biomarkers in a test sample from the subject, wherein the biomarkers correspond to the genes in Table 4 and the sample contains cancer cells. Differences or similarities in the expression of 73 biomarkers to the USC73 score are used for prognosis or classification of subjects with USC as poor or good survival groups. Scores of less than 9 indicated the subject will have an increased OS, and scores of less than 9 indicate that the subject will have less survivability than patients with a score less than 9.

In one aspect, the present invention provides a method for predicting prognosis in a subject having USC, comprising the following steps: a. Obtaining a subject biomarker expression profile in a subject sample of the 73 genes listed in Table 4; b. Obtaining a biomarker reference expression profile associated with prognosis, each of the target biomarker expression profile and the biomarker reference expression profile having 73 values, each value representing the expression level of the biomarker, each biomarker corresponds to one gene in Table 4; wherein a USC73 score less than 9 indicates the subject with have a increased OS and a score of less than 9 indicates that the subject will have less OS than subjects with scores of less than 9.

In another aspect, the prognostic and classification methods of the invention can be used to select a treatment. For example, the method can be used to select or identify subjects that may benefit from adjuvant chemotherapy in addition to surgical excision or only surgical excision. In some embodiments, a test value or composite score above the control value is predictive, for example, to be poor response or no gain from adjuvant therapy, while a composite score below the control value is for example, predicting good response or gain from adjuvant therapy. Accordingly, in one embodiment, the application provides a method of selecting a treatment for a subject with USC comprising the following steps: a. Classifying subjects with USC into poor response to treatment groups or good response to treatment groups according to the methods of the present invention, wherein patients with a score of less than 9 indicates the patient will have longer survival rates than patients with a score above 9 when treated with standard cancer therapies such as resection, chemotherapy, and radiation; and b. treating the patients with a score of less than 9 with standard chemotherapy.

In another embodiment, the invention is a method of prognosis or classification of a subject having USC, comprising the following steps: a. Determining the expression of 73 biomarkers in a test sample from the subject, wherein the biomarkers correspond to the genes in Table 4, and the test sample contains cancer cells, and b. Comparing the expression of 73 biomarkers in the test sample to the USC73 score. Differences or similarities in the expression of 73 biomarkers to the USC73 score are used for prognosis or classification of subjects with USC as poor or good survival groups, and c. determining the stage progression of USC (eg, early stage (I & II) or advanced stage (III & IV), and using the combination of the USC73 score and stage of USC for prognosis or classification of subjects with USC. For example, while high expression of the USC73 genes signatures (USC73_high patients) in combination with advanced stage (stage III or IV) USC correlates with poor prognosis and a 5-year OS of 0% to 11.6%, high expression of the USC73 genes signatures (USC73_high patients) in combination with early stage (stage I or II) USC correlates with intermediate prognosis and a 5-year OS of 45% to 82.7%, and low expression of the USC73 genes signatures (USC73_low patients) in combination with advanced stage (stage III or IV) USC correlates with intermediate prognosis and a 5-year OS of 48.9%.

Another aspect of the present invention provides kits useful for performing the prognostic tests described herein. The kit generally includes reagents and compositions for obtaining relative expression data for the 73 genes and any of the supplemental genes listed in Tables 2-4. As will be appreciated by those skilled in the art, the contents of the kit will vary depending on the means used to obtain relative expression information.

In one embodiment the kit comprises a labeled compound or agent capable of detecting a protein product or nucleic acid sequence in a sample, and a means for determining the amount of protein or mRNA in the sample (e.g., an antibody that binds to the protein or fragment thereof, or an oligonucleotide probe that binds to DNA or mRNA encoding the protein). The probe can be detectable, for example containing a detectable label such as a fluorophore, quantum dot, or isotope. The kit can also include instructions for interpreting the results obtained using the kit.

In some embodiments, the kit is an oligonucleotide-based kit, which can include, for example: (1) an oligonucleotide that hybridizes to the 73 identified biomarkers. The kit may also contain buffers, preservatives or protein stabilizers and the like. The kit can further include components necessary to detect the expression levels of the 73 biomarkers, including but not limited to a detectable label (e.g., an enzyme or a substrate). The kit can also include a control sample or series of control samples that can be assayed and compared to the test sample. Each component of the kit can be enclosed in a separate container, and all of the various containers are in a single package, with instructions for interpreting the results of the assay performed using the kit.

A further aspect provides computer implemented products, computer readable mediums and computer systems that are useful for the methods described herein.

### EXAMPLES

### Materials and Methods

### Study Design and Patients

The TCGA USC cohort (n=58) level 3, log2 transformed RNAseq data were obtained through the UCSC Xena data portal. The validation cohort consists of USC patients from the AU Medical Center. Data and sample collection were conducted through a retrospective, consent-waived arm of the IRB-approved Biomarkers and Therapies of Cancer study. Patients diagnosed with USC between 1999 and 2017, >18 years of age, and with sufficient formalin-fixed paraffin embedded (FFPE) tissues were included in the study (n=67, median follow-up time: 2.97 years). Patient demographic information is presented in Table 1. For data analyses purposes, patient age was discretized into <60 years and ≥60 years, while pathologic stage was separated into early (stage I and II) versus advanced (stage III and IV). Overall survival was the clinical endpoint for this study.

TABLE 1
Multivariate Cox Proportional Hazard Analysis on demographics and USC73 score. Chi-squared p-values
compare USC73_low and USC73_high within the cohort. P-value (AU-TCGA) compares clinical/
demographic characteristics of the two cohorts. For all analyses, α = 0.05 and bolded p values are < 0.05.

| Clinical and | TCGA cohort (n = 58) | | | AU cohort (n = 67) | | | p-value |
|---|---|---|---|---|---|---|---|
| Demographic | USC73_low | USC73_high | | USC73_low | USC73_high | | (AU- |
| Variables | (n = 39) | (n = 19) | p-value | (n = 45) | (n = 22) | p-value | TCGA) |
| Age at diagnosis | | | | | | | |
| <60 | 5 (12.8%) | 3 (15.8%) | 0.008 | 9 (20%) | 2 (9.1%) | 1.1E−02 | 0.076 |
| ≥60 | 34 (87.2%) | 16 (84.2%) | | 34 (75.6%) | 19 (86.4%) | | |
| Unknown | 0 (0%) | 0 (0%) | | 2 (4.4%) | 1 (4.5%) | | |
| Race | | | | | | | |
| AA* | 12 (30.8%) | 5 (26.3%) | 0.054 | 25 (55.6%) | 16 (72.7%) | 3.4E−03 | 1.9E−09 |
| Caucasian | 22 (56.4%) | 12 (63.2%) | | 18 (40%) | 3 (13.6%) | | |
| Other/Unknown | 5 (12.8%) | 2 (10.5%) | | 2 (4.4%) | 3 (13.6%) | | |
| Stage | | | | | | | |
| Early (I/II) | 24 (61.5%) | 5 (26.3%) | 0.0004 | 27 (60%) | 8 (36.4%) | 1.0E−03 | 0.28 |
| Advanced (III/IV) | 15 (38.5%) | 14 (73.7%) | | 18 (40%) | 14 (63.6%) | | |

Expression Analysis with NanoString

FFPE blocks with adequate tumor content were identified through systematic review of H&E slides of all AU USC cases, and cores of tissue (2 mm diameter×4 mm depth) with >60% tumor nuclei, as determined by a board-certified pathologist, were removed and stored at 4° C. until used for RNA extraction. RNA extraction from FFPE tissue was performed using a high-throughput protocol developed by our laboratory. Briefly, FFPE cores were mechanically and chemically disrupted using Citrisolve, heat (58° C. and 65° C.), and stainless steel beads. Then, FFPE lysates underwent column-based RNA extraction. RNA sample quality and concentration were assessed by Agilent (Santa Clara, Calif.) Tapestation RNA Analyzer and ThermoFisher (Waltham, Mass.) Nanodrop prior to gene expression quantification on NanoString (Seattle, Wash.) nCounter. RNA was stored at −80° C. in 2D Matrix barcode tubes, with limited freeze-thaw cycles.

Gene expression was quantified using a Custom Code Set containing probes for the USC73 gene signature. RNA (100-200 ng) was loaded into hybridization reaction as per manufacturer's recommendation. Five housekeeping genes (HNRNPL, IPO8, MRPL19, TBP, and GAPDH) were used to normalize the NanoString data. TCGA FKPM data and normalized AU Nanostring data were combined and batch normalized using multiplicative normalization factors calculated with geometric means of samples, then genes.

### Primary Cell Lines and Analyses

Primary tumor tissues or ascites samples were harvested form consented patients aseptically in the clinics, digested with collagenase, and cultured as adherent cells in DMEM medium supplemented with a primary cell culture supplements kindly provided by Jinfiniti Precision Medicine, Inc. (Augusta, Ga.). Information about the patients and their tumor is presented in Table 2. Cell growth was measured with the Cell Counting Kit-8 (Jinfiniti Biotech, LLC, Augusta, Ga.) for 5 days. Migration rate was determined with by scratch assays. Cell cycle was analyzed with PI staining and FACS.

TABLE 2
Patient characteristics for primary cell cultures. Patient tumor tissue and data collected through
BAT Cancer study, prospective arm.

| | | USC | | | | | | | Overall |
|---|---|---|---|---|---|---|---|---|---|
| Study | Sample | 73 | Age at | | FIGO | Date of | Date of | Date of last | survival |
| ID | ID | status | Diagnosis | Race | Stage | Diagnosis | recurrence | follow up | status |
| BAT682 | CT88T | low | 66.6 | Black | IIIA | Mar. 26, 2018 | Aug. 1, 2018 | Mar. 1, 2019 | 1 |
| BAT687 | CT89T | low | 69.8 | White | IA | May 3, 2016 | Apr. 9, 2018 | Jul. 25, 2019 | 0 |
| BAT647 | CaFld46A | low | 56.9 | Black | IIIC | May 18, 2017 | Oct. 19, 2017 | Feb. 9, 2018 | 1 |
| BAT705 | CaFld58A | low | 64.2 | Black | IVB | Jan. 31, 2018 | Jun. 3, 2019 | Jun. 10, 2019 | 0 |
| BAT789 | CT106T | high | 62.1 | Black | IIIA | Aug. 6, 2018 | NA | Aug. 14, 2018 | 0 |
| BAT788 | CT105T | low | 60.7 | Black | IA | Aug. 9, 2018 | NA | Jun. 21, 2019 | 0 |
| BAT600 | CT38T, | high | 55.5 | Black | IVB | Apr. 7, 2017 | Jan. 16, 2019 | Jul. 9, 2019 | 0 |
| | CT39T | | | | | | | | |
| | CT45T, | | | | | | | | |
| BAT621 | CT46T, | high | 75.3 | Black | IIIC | Aug. 23, 2017 | NA | Jun. 25, 2019 | 0 |
| | CT47T | | | | | | | | |

### Tissue Microarray and Immunostaining

Tissue microarray was constructed with 2 mm tissue cores on the 3DHISTECH (Kalamazoo, Mich.) TMA Grandmaster 2.0. Immunostaining was performed using Biocare (Pacheco, Calif.) predilute rabbit monoclonal anti-Ki-67 primary antibody.

### Cell Growth Assay

Cellular growth was monitored for all cell lines in a 96-well format, and all measurements were performed in triplicate. Each well was initially seeded with 2000 cells in 100 μL of modified DMEM media, then allowed to grow for 120 hours. Growth was measured using Cell Counting Kit-8 (CCK8) reagent (4% v/v), and the A450 was measured upon reagent addition, at 4 hours, 8 hours, 24 hours, and every 24 hours after that. Corresponding phase-contrast images were obtained to confirm the colorimetric data.

### Cellular Migration

Scratch assays were performed in 6-well plates. Cells were seeded at 2×10⁵ cells per well and allowed to proliferate for 24 hours. Then, a cross-pattern scratch was created using a P200 micropipette tip, providing quadruplicates for quantification. The cells are monitored and imaged at 40× magnification at regular intervals over the course of 30 hours. The scratch width is measured and normalized to initial scratch width. Migration rates are calculated by performing linear regression analysis for each cell line and extracting the slope of the line, which represents normalized migration distance over time. Both growth and migration rates are compared by two-sample t-test, with cell lines binned into either USC73_low or USC73_high groups.

### Cell Cycle Analysis

Adherent cells were trypsinized into single cell suspensions, then 2×10⁶ cells were exposed to BD PI/RNase Staining Buffer at room temperature for 30 minutes before cell sorting using the BD FACSCalibur flow cytometer. Detection of PI was at 495 nm in at least 10⁴ gated events. Fluorescence-activated cell sorting files were processed, gated, and analyzed using the BD FACS software.

### Immunostaining

Antigen retrieval was performed in Tris-EDTA buffer pH9, and slides were microwaved for 4 minutes on high (until boiling), then for 11 minutes at 20% power. Slides were blocked in 5% goat serum for 30 minutes at room temperature, followed by peroxide blocking in 3% H₂O₂ for 10 minutes at room temperature. Primary incubation in 1:100 anti-Ki-67 antibody occurred overnight at 4° C. Slides were then exposed to Biocare MACH2 Anti Rabbit IgG secondary antibody. Between each step, slides were washed in TBS-T (0.05% Tween-20).

Slides were digitized at 40× resolution, and the 3DHistech Case Viewer and the QuadCenter analysis add-on were used for quantification of immunostaining. Settings for training detection: Nuclear detection blur is 15, with radius 3-8 um and minimum area of 10. Nucleus filters include minimum intensity and contrast of 60. A staining intensity score of 3 has range of 0 to 70, 2 is 70 to 120, 1 is 120 to 200, and 0 is 200 to 255. H-score is used in future analyses to represent intensity and spread of staining.

### Statistical Analyses

Survival analysis was done using the Cox proportional hazard method. The hazard ratio (HR) and log rank test p-value were used to rank the genes. USC73 score was calculated using elastic net regression. Pathway enrichment analyses were conducted in StringDB. All statistical analyses were performed using the R language and environment for statistical computing (R version 3.5.1; R Foundation for Statistical Computing).

Univariate Cox regression analysis was used to identify a gene list significantly associated with OS. Expression data for these 73 genes in the TCGA was used to train an LO regularized (ridge) multivariate Cox model to calculate the USC73 score (‘glmnet’ package, alpha=0). The USC73 score was used to split the patients into two subsets at the 67% percentile. Samples with lower score were designated USC73_low and samples with higher score were designated USC73_high. USC73 status was evaluated as a univariate variable to assess survival differences.

Chi-squared analysis was performed to assess-1) whether the TCGA and AU cohorts had significant differences in their distributions of categorical covariates, and 2) whether the USC73_low and high groups in each cohort had significant differences in their distributions of categorical covariates. Univariate Cox proportional hazard models were made for each variable (USC73, stage, age at diagnosis, race, and treatment), and variables that significantly predicted OS (USC73 and stage, along with treatment in TCGA cohort) were included as part of a multivariate Cox proportional hazard models.

FPKM TCGA data and housekeeping gene-normalized AU Nanostring count data were combined into a parent data matrix, and these 2 data sets were batch normalized with each other using multiplicative normalization factors calculated with geometric means of first samples, then genes. To harmonize TCGA's normalized, not log-transformed FKPM values and AU's housekeeping gene-normalized and background thresholded NanoString count data, sample normalization constants were computed by dividing samples' gene expression by the geometric mean of all genes' expression in that sample. Then, gene normalization constants were computed by dividing individual gene's expression values by the geometric means of each sample's expression values.

Hierarchical clustering of cell line gene expression was calculated using Manhattan distance and the average method.

All statistical tests were two-tailed unless otherwise noted and a p<0.05 was considered statistically significant.

### Example 1: Selection of Prognostic Genes Using the TCGA RNAseq Data

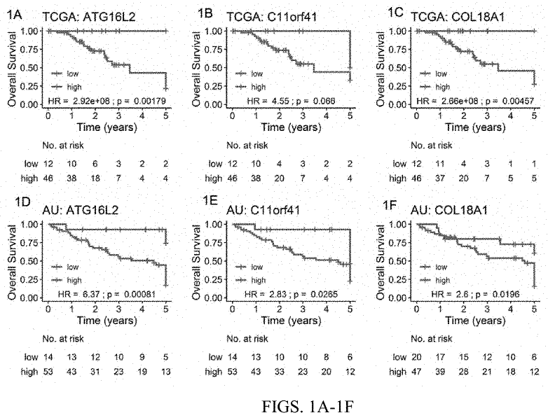

Cox proportional hazard analysis was carried out for each of the 20,530 genes in the TCGA transcriptomic dataset. A combination of HR and p-value (HR>10⁸, p<0.01) was used to select the top 105 genes, which were further reduced to 73 genes based on gene functions and potential relevance to cancer (Table 3). High expressers of these 73 genes have greatly lower 5-year survival in comparison to low expressers. FIG. 1 shows the Kaplan Meier survival curves for representative genes.

TABLE 3
USC73 gene signature genes selected in the discovery
(TCGA) cohort. Threshold for discretization of high and
lower expressers is shown as “cutoff (% ile)” column. The
overall p-value reported for each univariate model is the
log-rank p-value. On discretized univariate Cox analysis,
genes with the highest hazard ratios were included.

| | Cutoff | | | | Log rank |
|---|---|---|---|---|---|
| Gene | (% ile) | High n | Low n | HR | p-val |
| ACRC | 30 | 40 | 18 | 3.15E+08 | 0.001 |
| AG2 | 20 | 46 | 12 | 3.05E+08 | 0.002 |
| ATG16L2 | 20 | 46 | 12 | 2.74E+08 | 0.005 |
| C10orf47 | 20 | 46 | 12 | 2.66E+08 | 0.007 |
| C11orf41 | 20 | 46 | 12 | 2.63E+08 | 0.008 |
| C17orf70 | 30 | 40 | 18 | 3.19E+08 | 0.002 |
| C1orf126 | 20 | 46 | 12 | 2.91E+08 | 0.003 |
| C3orf66 | 20 | 46 | 12 | 3.13E+08 | 0.003 |
| C8orf45 | 20 | 46 | 12 | 2.81E+08 | 0.005 |
| CNOT1 | 20 | 46 | 12 | 2.89E+08 | 0.004 |
| COL18A1 | 20 | 46 | 12 | 2.79E+08 | 0.004 |
| CUBN | 30 | 40 | 18 | 3.29E+08 | 9.39E−04 |
| DENND2A | 20 | 46 | 12 | 2.92E+08 | 0.004 |
| DKK1 | 20 | 46 | 12 | 2.69E+08 | 0.006 |
| DOK4 | 30 | 40 | 18 | 3.60E+08 | 4.01E−04 |
| F3 | 30 | 40 | 18 | 3.29E+08 | 0.001 |
| FLJ23867 | 20 | 46 | 12 | 3.12E+08 | 0.002 |
| GALNTL2 | 20 | 46 | 12 | 2.79E+08 | 0.005 |
| GALNTL4 | 20 | 46 | 12 | 3.22E+08 | 0.002 |
| GNAL | 20 | 46 | 12 | 2.78E+08 | 0.006 |
| GPR111 | 30 | 40 | 18 | 2.93E+08 | 0.003 |
| GRIA3 | 20 | 46 | 12 | 2.67E+08 | 0.008 |
| HGS | 40 | 35 | 23 | 4.48E+08 | 5.73E−05 |
| HS3ST2 | 20 | 46 | 12 | 2.75E+08 | 0.005 |
| IBTK | 20 | 46 | 12 | 3.07E+08 | 0.003 |
| IL1R2 | 20 | 46 | 12 | 2.74E+08 | 0.005 |
| ITGA10 | 20 | 46 | 12 | 2.63E+08 | 0.008 |
| KCNE4 | 20 | 46 | 12 | 2.84E+08 | 0.004 |
| LOC728264 | 30 | 40 | 18 | 3.18E+08 | 0.001 |
| LY6H | 20 | 46 | 12 | 2.82E+08 | 0.005 |
| MC1R | 20 | 46 | 12 | 2.73E+08 | 0.006 |
| MEIS3 | 20 | 46 | 12 | 2.62E+08 | 0.008 |
| MST1R | 20 | 46 | 12 | 2.90E+08 | 0.003 |
| NCOA7 | 30 | 40 | 18 | 3.59E+08 | 4.79E−04 |
| NPAS2 | 30 | 40 | 18 | 3.59E+08 | 4.43E−04 |
| OBFC2A | 30 | 40 | 18 | 3.40E+08 | 7.33E−04 |
| OVGP1 | 20 | 46 | 12 | 2.80E+08 | 0.006 |
| PAPPA | 20 | 46 | 12 | 2.66E+08 | 0.008 |
| PHLDA2 | 30 | 40 | 18 | 4.39E+08 | 1.68E−04 |
| RBMS2 | 20 | 46 | 12 | 2.72E+08 | 0.007 |
| S100A10 | 20 | 46 | 12 | 2.64E+08 | 0.008 |
| S100A11 | 30 | 40 | 18 | 3.63E+08 | 3.53E−04 |
| S100A6 | 20 | 46 | 12 | 2.62E+08 | 0.009 |
| SAA4 | 40 | 35 | 23 | 1.86E+09 | 2.30E−06 |
| SH2D3A | 30 | 40 | 18 | 3.04E+08 | 0.002 |
| TXK | 20 | 46 | 12 | 2.67E+08 | 0.007 |
| VIL1 | 30 | 40 | 18 | 3.28E+08 | 9.42E−04 |
| WNT7B | 20 | 46 | 12 | 2.63E+08 | 0.009 |
| ZNF69 | 30 | 40 | 18 | 3.18E+08 | 0.001 |

### Example 2: USC73 Score Computed Using Elastic Net Regression

While each of the 73 genes has good prognostic potential, a gene signature is expected to have more robust and potentially better prognostic value and is more likely translatable to clinical practice. Therefore, gene expression values were combined into a linear predictor value for each patient using elastic net regression performed on TCGA gene expression data. The computed score, termed USC73, uses different weights for each gene as reported in Table 4.

TABLE 4
USC73 ridge model weights.

| Gene | Weights | Gene | Weights |
|---|---|---|---|
| CNOT1 | 0.086 | C1orf106 | 0.021 |
| ACRC | 0.072 | MEIS3 | 0.021 |
| HGS | 0.066 | GALNTL2 | 0.020 |
| C8orf45 | 0.058 | GALNTL4 | 0.019 |
| IBTK | 0.054 | WNT7B | 0.018 |
| PHLDA2 | 0.051 | DENND2A | 0.018 |
| C1orf126 | 0.050 | IER3 | 0.016 |
| FLJ35776 | 0.049 | MYEOV | 0.015 |
| BTBD16 | 0.046 | S100A10 | 0.014 |
| MC1R | 0.045 | GNAL | 0.014 |
| RBMS2 | 0.042 | MST1R | 0.012 |
| IL1R2 | 0.041 | KCNE4 | 0.012 |
| COL18A1 | 0.039 | CUBN | 0.011 |
| CHRNA10 | 0.039 | TAL1 | 0.011 |
| S100A6 | 0.037 | MMP10 | 0.011 |
| S100A11 | 0.037 | GPR124 | 0.011 |
| EIF2B2 | 0.035 | WDR17 | 0.010 |
| OBFC2A | 0.034 | HABP2 | 0.010 |
| C10orf47 | 0.034 | GRIA3 | 0.009 |
| LOC728264 | 0.033 | COL4A4 | 0.008 |
| ATG16L2 | 0.033 | TXK | 0.007 |
| C17orf70 | 0.031 | GPR111 | 0.007 |
| COL1A1 | 0.029 | HS3ST2 | 0.007 |
| RHOV | 0.029 | SLC6A13 | 0.007 |
| DOK4 | 0.028 | DKK1 | 0.005 |
| FLJ23867 | 0.027 | PADI1 | 0.004 |
| LIPG | 0.027 | LY6H | 0.004 |
| ZNF69 | 0.027 | C2CD4A | 0.004 |
| C11orf41 | 0.026 | VIL1 | 0.003 |
| C11orf9 | 0.026 | AG2 | 0.003 |
| ERBB2 | 0.026 | IL6 | 0.003 |
| C3orf66 | 0.025 | OVGP1 | 0.002 |
| SAA4 | 0.025 | NCOA7 | 0.002 |
| NPAS2 | 0.025 | ITGA10 | 0.000 |
| SH2D3A | 0.024 | C12orf27 | −0.001 |
| CLDN14 | 0.024 | F3 | −0.003 |
| PAPPA | 0.021 | | |

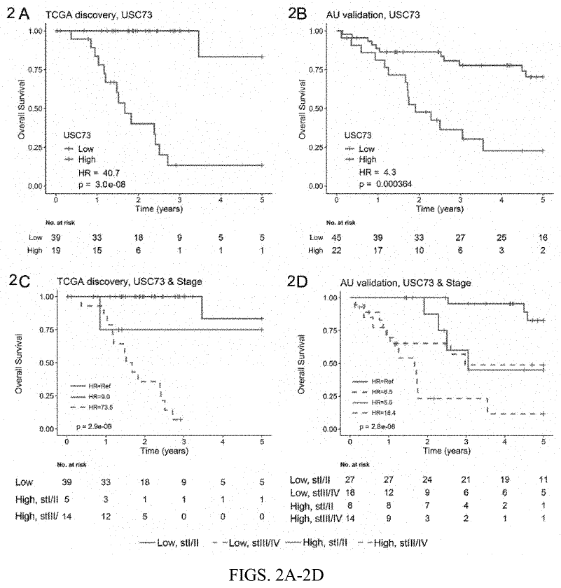

The USC73 score ranges from 7.3 to 10.3 (median of 8.68). The score was used to separate patients into two groups at the 67^{th} percentile. USC73_high patients have poor prognosis (5-year OS=13.3%, median survival time=1.67 years) while USC73_low patients have drastically improved prognosis (5-year OS=83.3%, median survival time >5 years) (HR=40.1, p=3×10⁻⁸, FIG. 2A).

### Example 3: Validation of the USC73 Gene Signature

To validate the USC73 gene signature, the expression of the USC73 genes was quantified in archived FFPE tissues of USC patients treated in the Augusta area from 1999 to 2017 using the NanoString single-molecule counting technology. The NanoString data were harmonized with TCGA RNAseq expression data through multiplicative normalization constants. In the AU validation cohort, 40 of the 73 genes individually showed statistically significant survival differences on Cox proportional hazard analysis and 12 additional genes showed survival differences with trending significance (Table 5).

TABLE 5
USC73 gene signature genes that remain individually prognostic in
the validation (AU) cohort. Threshold for discretization of high and
lower expressers is shown as “cutoff (% ile)” column. The overall
p-value reported for each univariate model is the log-rank p-value.
For all analyses, α = 0.05.

| | Cutoff | | | | Log rank |
|---|---|---|---|---|---|
| Gene | (% ile) | High n | Low n | HR | p-val |
| ACRC | 60 | 25 | 38 | 2.61 | 0.008 |
| AG2 | 40 | 38 | 25 | 2.94 | 0.006 |
| ATG16L2 | 20 | 50 | 13 | 13.57 | 7.24E−05 |
| BTBD16 | 60 | 25 | 38 | 2.82 | 0.005 |
| C11orf41 | 20 | 50 | 13 | 3.27 | 0.012 |
| C11orf9 | 30 | 44 | 19 | 4.12 | 0.002 |
| C17orf70 | 60 | 25 | 38 | 1.80 | 0.099 |
| C1orf106 | 20 | 50 | 13 | 2.83 | 0.048 |
| C1orf126 | 30 | 44 | 19 | 2.45 | 0.026 |
| C2CD4A | 30 | 44 | 19 | 2.84 | 0.012 |
| C3orf66 | 50 | 31 | 32 | 2.02 | 0.052 |
| C8orf45 | 70 | 19 | 44 | 2.55 | 0.012 |
| CALML6 | 40 | 38 | 25 | 2.22 | 0.032 |
| CHRNA10 | 50 | 31 | 32 | 2.30 | 0.021 |
| CLDN14 | 50 | 31 | 32 | 2.57 | 0.009 |
| COL18A1 | 30 | 44 | 19 | 3.27 | 0.006 |
| COL1A1 | 40 | 38 | 25 | 1.93 | 0.079 |
| COL4A4 | 40 | 38 | 25 | 2.70 | 0.010 |
| CUBN | 60 | 25 | 38 | 2.60 | 0.008 |
| DENND2A | 30 | 44 | 19 | 4.06 | 0.001 |
| DKK1 | 50 | 31 | 32 | 2.12 | 0.037 |
| FLJ35776 | 20 | 50 | 13 | 2.89 | 0.026 |
| GALNTL2 | 50 | 31 | 32 | 2.87 | 0.004 |
| GNAL | 50 | 31 | 32 | 2.63 | 0.008 |
| GPR124 | 70 | 19 | 44 | 2.40 | 0.021 |
| GRIA3 | 60 | 25 | 38 | 2.19 | 0.032 |
| HS3ST2 | 50 | 31 | 32 | 3.03 | 0.003 |
| IL6 | 20 | 50 | 13 | 3.84 | 0.009 |
| ITGA10 | 70 | 19 | 44 | 2.21 | 0.035 |
| LIPG | 20 | 50 | 13 | 3.29 | 0.022 |
| LOC728264 | 60 | 25 | 38 | 2.35 | 0.019 |
| LY6H | 50 | 31 | 32 | 2.19 | 0.030 |
| MC1R | 50 | 31 | 32 | 2.80 | 0.005 |
| MEIS3 | 40 | 38 | 25 | 2.17 | 0.043 |
| MST1R | 20 | 50 | 13 | 2.74 | 0.057 |
| MYEOV | 60 | 25 | 38 | 1.94 | 0.065 |
| NCOA7 | 30 | 44 | 19 | 2.39 | 0.038 |
| PAPPA | 50 | 31 | 32 | 2.76 | 0.006 |
| PHLDA2 | 20 | 50 | 13 | 4.45 | 0.011 |
| RBMS2 | 20 | 50 | 13 | 3.44 | 0.016 |
| RHOV | 20 | 50 | 13 | 2.51 | 0.041 |
| SAA4 | 40 | 38 | 25 | 1.98 | 0.068 |
| SLC23A3 | 70 | 19 | 44 | 1.99 | 0.066 |
| SLC6A13 | 50 | 31 | 32 | 2.27 | 0.023 |
| TAL1 | 70 | 19 | 44 | 2.17 | 0.039 |
| VIL1 | 60 | 25 | 38 | 1.84 | 0.089 |
| WDR17 | 30 | 44 | 19 | 2.82 | 0.010 |
| WNT7B | 30 | 44 | 19 | 2.45 | 0.026 |
| ZNF69 | 60 | 25 | 38 | 1.81 | 0.096 |

The model trained on TCGA data was used to calculate USC73 score for each patient in the AU validation dataset. The USC73 score ranges from 7.2 to 12.4 (median of 10.1). The AU patients were similarly separated into two groups at the 67^{th} percentile of USC73 score. Similar to the TCGA cohort, the 5-year survival is 22.7% and 70.4% for the USC73_high and low groups, respectively (HR=4.3, p=0.00036), thereby validating the USC73 gene signature as a prognostic biomarker in an independent USC cohort. The median survival time is greater than 5 years for the USC73_low patients and 1.91 years for the USC73_high patients in the AU validation cohort (FIG. 2B).

### Example 4: USC73 Predicts Survival Independent of Stage

Clinical and demographic characteristics of TCGA and AU cohorts are shown in Table 1. The AU cohort has a significantly higher proportion of African American patients overall. TCGA USC73_high patients tend to be advanced stage (stage III or IV). AU USC73_high patients tend to be more than 60 years of age at diagnosis, African American, and of advanced stage (p=1.1×10⁻², 3.4×10⁻³, and 1.0×10⁻³, respectively). Cox proportional hazard analysis for each covariate (Table 6) showed that advanced stage is associated with poor prognosis in the TCGA cohort (HR=7.8 and p=9.1×10⁻⁴) and the AU cohort (HR=5.4, p=4.9×10⁻⁵) while age and race are not associated with survival. Multivariate analysis including all significant covariates showed that USC73 influences survival independent of stage in both the TCGA (HR=30.5, p=0.001) and the AU cohort (HR=3.4, p=0.003). Despite the demographic differences between the two cohorts, USC73 predicts overall survival in both cohorts, demonstrating the generalizability of the USC73 score across diverse patient groups.

TABLE 6
Univariate Cox proportional hazard analysis of patient
demographics based on USC73 predictor score and cohort. The
overall p-value reported for each univariate model is the log-rank
p-value. For all analyses, α = 0.05 and bolded p values are <0.05.

| Clinical and | | | | |
|---|---|---|---|---|
| Demographic | TCGA cohort (n = 58) | | AU cohort (n = 67) | |
| Variables | HR | p-value | HR | p-value |
| USC73 status | Reference | 3.0E−08 | Reference | 3.6E−04 |
| low | 40.7 | | 4.3 | |
| high | | | | |
| Age at diagnosis | 1.0 | 0.3 | 1.0 | 0.6 |
| Race | Reference | 0.9 | Reference | 0.2 |
| African | 0.9 | | 0.4 | |
| American | 1.2 | | 1.2 | |
| Caucasian | | | | |
| Other/Unknown | | | | |
| Stage | — | 9.1E−04 | — | 4.9E−05 |
| Early (I/II) | 7.8 | | 5.4 | |
| Advanced | | | | |
| (III/IV) | | | | |
| Treatment | Reference | 3.0E−03 | Reference | 0.07 |
| None | — | | 0.3 | |
| Chemo & | — | | 0.4 | |
| Radiation | 5.4 | | 0.8 | |
| Chemotherapy | | | | |
| Radiation | | | | |

### Example 5: USC73 and Stage Together Provide Better Prognosis

Using a combination of USC73 score and stage, four patient groups could be defined. Kaplan Meier survival curves for the four groups are shown in FIGS. 2C and 2D for the TCGA and AU cohorts, respectively. We decided to not split the TCGA USC73_low patients based on stage because only one patient in the group did not survive within the follow-up period. However, among USC73_high patients in the TCGA, advanced stage clearly has worse prognosis (p<2.9×10⁻⁸) (FIG. 2C). In TCGA, the 5-year OS rate is 83.3% in the USC73_low reference group, while the worst survival group consists of patients with USC73_high and advanced stage with a 5-year OS of 0%. The early stage patients with USC73_high have intermediate prognosis (HR=9.0 and 5-year OS=75%).

In the AU validation cohort, advanced stage is associated with worse prognosis in both USC_high and USC73_low groups (p=2.8×10⁻⁶). The 5-year OS rate is 82.7% in the early stage USC73_low (reference group), while the worst survival group consists of patients with USC73_high and advanced stage with a 5-year OS of only 11.6%. The HR between the worst prognosis group (advanced stage USC73_high) and the best survival (reference) group is 18.4 (FIG. 2D). The advanced stage USC73_low group and early stage USC73_high group have intermediate prognosis (HR=6.5 and 5.5 respectively, and 5-year OS=48.9% and 45.0% respectively).

### Example 6: USC73_High Tumors have Higher Proliferation Rates

KEGG Pathway analysis of differentially expressed genes between USC73_high and low patients shows enrichment of “Base Excision Repair” and “DNA Replication” pathways (p=0.017 for both, Table 7). Previous molecular studies have also reported cellular proliferation and DNA repair as important pathways in USC pathogenesis, in agreement with our analysis.

TABLE 7
Enriched KEGG Pathways in differentially expressed genes between USC73_high
and USC73_low patients in TCGA cohort. 73 genes were significantly differentially
expressed at a genome-level p-value adjustment (Benjamini-Hochberg method),
and these 73 genes were inputted into StringDB, resulting in the following significant
KEGG pathways. Functions listed extracted from StringDB legend output.

| | | | | Matching | |
|---|---|---|---|---|---|
| | | | | proteins | |
| | Observed/ | False | Matching proteins | in your | |
| KEGG | background | discovery | in your network | network | |
| Pathway | gene count | rate | (IDs) | (labels) | Function |
| DNA | 3/36 | 0.0173 | ENSP00000305480 | FEN1 | Flap |
| replication | | | | | endonuclease 1; |
| | | | | | Structure-specific |
| | | | | | nuclease with |
| | | | | | 5′-flap |
| | | | | | endonuclease |
| | | | | | and 5′-3′ |
| | | | | | exonuclease |
| | | | | | activities |
| | | | | | involved in |
| | | | | | DNA |
| | | | | | replication and |
| | | | | | repair. During |
| | | | | | DNA |
| | | | | | replication, |
| | | | | | cleaves the 5′- |
| | | | | | overhanging |
| | | | | | flap structure |
| | | | | | that is generated |
| | | | | | by displacement |
| | | | | | synthesis when |
| | | | | | DNA |
| | | | | | polymerase |
| | | | | | encounters the |
| | | | | | 5′-end of a |
| | | | | | downstream |
| | | | | | Okazaki |
| | | | | | fragment. It |
| | | | | | enters the flap |
| | | | | | from the 5′-end |
| | | | | | and then tracks |
| | | | | | to cleave the |
| | | | | | flap base, |
| | | | | | leaving a nick |
| | | | | | for ligation. |
| | | | | | Also involved |
| | | | | | in the long |
| | | | | | patch base |
| | | | | | excision repair |
| | | | | | (LP-BER) |
| | | | | | pathway, by |
| | | | | | cleaving within the |
| | | | | | apurinic/apyrim |
| | | | | | idinic (AP) site- |
| | | | | | terminated flap. |
| | | | | | (380 aa) |
| | | | ENSP00000369411 | RFC1 | Replication |
| | | | | | factor C subunit |
| | | | | | 1; The |
| | | | | | elongation of |
| | | | | | primed DNA |
| | | | | | templates by |
| | | | | | DNA |
| | | | | | polymerase |
| | | | | | delta and |
| | | | | | epsilon requires |
| | | | | | the action of the |
| | | | | | accessory |
| | | | | | proteins PCNA |
| | | | | | and activator 1. |
| | | | | | This subunit |
| | | | | | binds to the |
| | | | | | primer-template |
| | | | | | junction. Binds |
| | | | | | the PO-B |
| | | | | | transcription |
| | | | | | element as well |
| | | | | | as other GA |
| | | | | | rich DNA |
| | | | | | sequences. |
| | | | | | Could play a |
| | | | | | role in DNA |
| | | | | | transcription |
| | | | | | regulation as |
| | | | | | well as DNA |
| | | | | | replication |
| | | | | | and/or repair. |
| | | | | | Can bind single- |
| | | | | | or double- |
| | | | | | stranded DNA; |
| | | | | | AAA ATPases |
| | | | | | (1148 aa) |
| | | | ENSP00000371321 | RFC3 | Replication |
| | | | | | factor C subunit |
| | | | | | 3; The |
| | | | | | elongation of |
| | | | | | primed DNA |
| | | | | | templates by DNA |
| | | | | | polymerase |
| | | | | | delta and |
| | | | | | epsilon requires |
| | | | | | the action of the |
| | | | | | accessory |
| | | | | | proteins |
| | | | | | proliferating |
| | | | | | cell nuclear |
| | | | | | antigen (PCNA) |
| | | | | | and activator 1; |
| | | | | | AAA ATPases |
| | | | | | (356 aa) |
| Base | 3/33 | 0.0173 | ENSP00000242576 | FEN1 | Flap |
| excision | | | | | endonuclease 1; |
| repair | | | | | Structure-specific |
| | | | | | nuclease with |
| | | | | | 5′-flap |
| | | | | | endonuclease |
| | | | | | and 5′-3′ |
| | | | | | exonuclease |
| | | | | | activities |
| | | | | | involved in DNA |
| | | | | | replication and |
| | | | | | repair. During |
| | | | | | DNA replication, |
| | | | | | cleaves the 5′- |
| | | | | | overhanging |
| | | | | | flap structure |
| | | | | | that is generated |
| | | | | | by displacement |
| | | | | | synthesis when |
| | | | | | DNA polymerase |
| | | | | | encounters the |
| | | | | | 5′-end of a |
| | | | | | downstream |
| | | | | | Okazaki |
| | | | | | fragment. It |
| | | | | | enters the flap |
| | | | | | from the 5′-end |
| | | | | | and then tracks |
| | | | | | to cleave the |
| | | | | | flap base, |
| | | | | | leaving a nick |
| | | | | | for ligation. |
| | | | | | Also involved |
| | | | | | in the long |
| | | | | | patch base |
| | | | | | excision repair |
| | | | | | (LP-BER) |
| | | | | | pathway, by |
| | | | | | cleaving within the |
| | | | | | apurinic/apyrim |
| | | | | | idinic (AP) site- |
| | | | | | terminated flap. |
| | | | | | (380 aa) |
| | | | ENSP00000305480 | PARP1 | Poly [ADP- |
| | | | | | ribose] |
| | | | | | polymerase 1; |
| | | | | | Involved in the |
| | | | | | base excision |
| | | | | | repair (BER) |
| | | | | | pathway, by |
| | | | | | catalyzing the |
| | | | | | poly(ADP- |
| | | | | | ribosyl)ation of |
| | | | | | a limited |
| | | | | | number of acceptor |
| | | | | | proteins involved in |
| | | | | | chromatin |
| | | | | | architecture and |
| | | | | | in DNA metabolism. |
| | | | | | This modification |
| | | | | | follows DNA |
| | | | | | damages and |
| | | | | | appears as an |
| | | | | | obligatory step in a |
| | | | | | detection/signaling |
| | | | | | pathway |
| | | | | | leading to the |
| | | | | | reparation of |
| | | | | | DNA strand breaks. |
| | | | | | Mediates the |
| | | | | | poly(ADP- |
| | | | | | ribosyl)ation of |
| | | | | | APLF and CHFR. |
| | | | | | Positively |
| | | | | | regulates the |
| | | | | | transcription of |
| | | | | | MTUS1 and |
| | | | | | negatively |
| | | | | | regulates the |
| | | | | | transcription of |
| | | | | | MTUS2/TIP150. |
| | | | | | With EEF1A1 |
| | | | | | and TXK, forms |
| | | | | | a complex [ . . . ] |
| | | | | | (1014 aa) |
| | | | ENSP00000355759 | UNG | Uracil-DNA |
| | | | | | glycosylase; |
| | | | | | Excises uracil |
| | | | | | residues from |
| | | | | | the DNA which |
| | | | | | can arise as a |
| | | | | | result of |
| | | | | | misincorporation |
| | | | | | of dUMP |
| | | | | | residues by DNA |
| | | | | | polymerase or due |
| | | | | | to deamination of |
| | | | | | cytosine; |
| | | | | | Belongs to the |
| | | | | | uracil-DNA |
| | | | | | glycosylase |
| | | | | | (UDG) superfamily. |
| | | | | | UNG family |
| | | | | | (313 aa) |

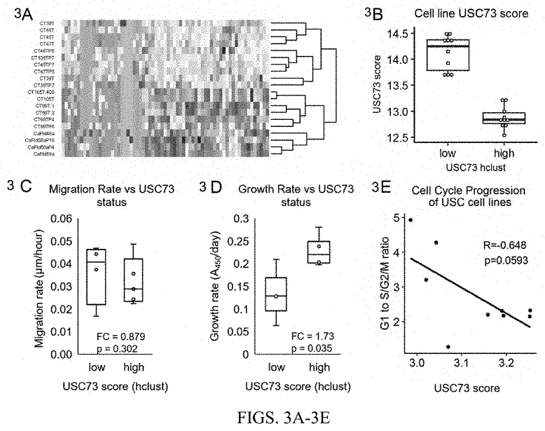

To gain functional insights into the tumors associated with USC73 gene signature, cell growth and migration phenotypes were investigated in 10 USC primary cell lines established in this study. Gene signature was determined using the same NanoString assay. Hierarchical clustering of the gene expression heatmap (FIG. 3A) and the USC73 score for each patient (FIG. 3B) suggested two groups of cell lines corresponding to USC73_high and USC73_low expression profiles. Scratch assays showed no significant difference in migration rate between USC73_high and low cell lines (FIG. 3C). However, USC73_high cell lines had 1.7-fold higher growth rate (p=0.035, FIG. 3D). The ratio of cells in G1 versus S/G2/M phase were quantified with propidium iodide staining (FIG. 3E). Cell lines with higher USC73 scores have decreased G1 to S/G2/M ratio and USC73_high cell lines also undergo cellular division more regularly than USC73_low cell lines (R=−0.648). This increased cell cycle progression amongst cell lines with higher USC73 scores likely contributes to higher growth rate.

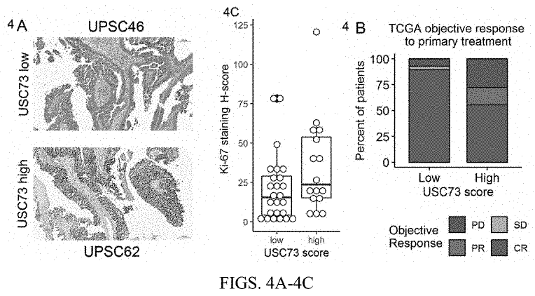

A tissue microarray (TMA) was constructed from FFPE blocks for the AU patients and immunostained for Ki-67, a marker of cellular proliferation (FIG. 4A). USC73_high tumors had, on average, 1.5-fold greater H score, representing staining intensity and proportion of positive cells, than USC73_low tumors (FIG. 4B), with trending significance on two sample t-test (p=0.054). These results suggest that the poor survival associated with USC73_high patients may be, at least partially, due to their tumor's increased cellular proliferation rate.

### Example 7: USC73_High Patients have Decreased Objective Response to Primary Treatment

In the TCGA cohort, the complete response rate is higher for USC73_low patients (89.3%) than USC73_high patients (55.6%) and the proportion of progressive disease is lower in USC73_low patients (7.1%) than USC73_high patients (27.8%) (p=0.018, FIG. 4C). The difference in objective response suggests that the USC73 gene signature may be associated with treatment response and may serve as a therapeutic biomarker in addition to being a prognostic biomarker.

USC is a rare subtype of uterine cancer and therefore most studies have very small numbers of patients, often admixed with other uterine cancer histologies. Our sample size is amongst the larger cohorts with genomic or molecular data reported for USC. This study used the TCGA cohort as a discovery dataset and a local dataset as validation to develop a transcriptomic gene signature that can predict OS.

Many of the 73 genes have functions relevant to cancer and the most important ones include MSTIR, IER3, and C17orf70. MSTIR is an oncogene regulating cell survival and migration. IER3 is responsible for increased ERK downstream signaling, confers increased cancer cell survival, and has been associated with increased chemosensitivity in multiple cancers. C17orf70 induces chromosomal instability and is associated with sensitivity to DNA cross-linking agents. The gene signature of the present invention consists of 73 genes, thus termed USC73, and is a predictor of OS independent of other clinicopathological variables, namely stage. The 5-year survival difference is very large between USC73_high and USC73_low (HR=40.7 and 4.3 in the TCGA and AU cohorts). Among the tested clinicopathologic variables, stage is the only one that predicts USC survival, and importantly, USC73 and stage can be combined to give better stratification of patients into different survival groups. With HRs of 73.5 and 18.4 (TCGA and AU, respectively) between the USC73_high, advanced stage patients and the reference groups, the combined prognostic model is highly predictive and clinically relevant for USC patients.

To gain insight into the functional implications of the USC73 signature, we investigated different molecular and cellular characteristics including cellular proliferation, cell cycle and migration. For these studies, we established 10 primary cancer cell lines from USC patients and determined their USC73 profiles. Our results show that the USC73_high signature was associated with increased cell cycle progression and growth rate, consistent with their expected poor survival and corroborating reports of cell cycle proteins and growth receptors as prognostic biomarkers in USC.

It is important to point out that prognostic prediction by biomarkers is certainly associated with the treatments that patients have received and can change as treatment changes. In the case of USC, treatment for almost all patients has been surgical resection in combination with chemotherapy with or without radiotherapy, hence divergence in patient prognosis is hypothesized to be due to variation in patient response to the current standard therapy. This hypothesis is supported by the association between the USC73 status and objective response to primary therapy (FIG. 4). Furthermore, increased cellular proliferation has been implicated as a marker of chemotherapy response, particularly for cisplatin in uterine adenocarcinoma patients. Since proliferation is one of the mechanisms through which USC73 will be prognostic, USC73 is also a therapeutic response biomarker for USC, in addition to being a prognostic biomarker, evidenced by the association of USC73 score with objective response (FIG. 4C)

As a therapeutic biomarker, USC73 will be useful for patient care and future clinical trials. Immediately, USC73 score will tell patients and their physicians about their expected OS if they are treated with the standard therapies, which would have excellent outcome for those early stage patients with a low USC73 score. However, early stage patients with high USC73 score, advanced stage patients, and especially those advanced stage patients with high USC73 score have bad prognosis and alternative treatment options should be considered. Options may include changing standard chemotherapy combinations to other combinations in the upfront setting, instead of changing upon disease progression.

The USC73 signature may also prove very useful for future clinical trials in selecting patients who have worse and more homogeneous prognosis, hence increasing the power of the trials. The large heterogeneity in survival prognosis among USC patients, in addition to small sample size and poor selection of treatment regimen, is understandably a contributing factor to the lack of success in USC clinical trials.

Since USC73 can be assayed using the NanoString single molecule counting technology, it can be easily translated into clinical labs as a suitable RNA source is FFPE blocks, which are available for almost all patients who undergo hysterectomies as part of their standard care. Additionally the assay is highly reproducible and other similar assays such as the PAM50 breast cancer assay are already FDA-approved. Therefore, the path to move the assay from laboratory to bedside is relatively straightforward.

Some embodiments provide transcriptomic biomarkers associated with uterine serous carcinomas (USC), methods for the prognosis of USC and for predicting patient response to therapy.

### REFERENCES

1. Siegel R L, Miller K D, Jemal A: Cancer statistics, 2019. CA Cancer J

Clin 69:7-34, 2019
2. Del Carmen M G, Birrer M, Schorge J O: Uterine papillary serous cancer: a review of the literature. Gynecologic oncology 127:651-661, 2012
3. Le Gallo M, Bell D W: The emerging genomic landscape of endometrial cancer. Clinical chemistry 60:98-110, 2014
4. Naumann R W: Uterine papillary serous carcinoma: state of the state. Current oncology reports 10:505-511, 2008
5. Fader A N, Santin A D, Gehrig P A: Early stage uterine serous carcinoma:

management updates and genomic advances. Gynecologic oncology 129:244-250, 2013
6. Hong B, Le Gallo M, Bell D W: The mutational landscape of endometrial cancer. Current opinion in genetics & development 30:25-31, 2015
7 Jones N L, Xiu J, Reddy S K, et al: Identification of potential therapeutic targets by molecular profiling of 628 cases of uterine serous carcinoma. Gynecologic oncology 138:620-626, 2015
8. Fleming G F, Sill M W, Darcy K M, et al: Phase II trial of trastuzumab in women with advanced or recurrent, HER2-positive endometrial carcinoma: a Gynecologic Oncology Group study. Gynecologic oncology 116:15-20, 2010
9. Janku F, Wheler J J, Westin S N, et al: PI3K/AKT/mTOR inhibitors in patients with breast and gynecologic malignancies harboring PIK3CA mutations. Journal of clinical oncology 30:777, 2012
10. Gupta D, Gunter M J, Yang K, et al: Performance of serum CA125 as a prognostic biomarker in patients with uterine papillary serous carcinoma.

International Journal of Gynecological Cancer 21:529-534, 2011
11. Kallakury B, Ambros R A, Hayner-Buchan A M, et al: Cell proliferation-associated proteins in endometrial carcinomas, including papillary serous and endometrioid subtypes. International journal of gynecological pathology: official journal of the International Society of Gynecological

Pathologists 17:320-326, 1998
12. Hanahan D, Weinberg R A: Hallmarks of cancer: the next generation. cell 144:646-674, 2011
13. Zhang Y, Zhao D, Gong C, et al: Prognostic role of hormone receptors in endometrial cancer: a systematic review and meta-analysis. World journal of surgical oncology 13:208, 2015
14. Togami S, Sasajima Y, Oi T, et al: Clinicopathological and prognostic impact of human epidermal growth factor receptor type 2 (HER2) and hormone receptor expression in uterine papillary serous carcinoma. Cancer Science 103:926-932, 2012
15. Busch E L, Crous-Bou M, Prescott J, et al: Endometrial cancer risk factors, hormone receptors, and mortality prediction. Cancer Epidemiology and Prevention Biomarkers, 2017
16. Slomovitz B M, Broaddus R R, Burke T W, et al: Her-2/neu Overexpression and Amplification in Uterine Papillary Serous

Carcinoma. Journal of Clinical Oncology 22:3126-3132, 2004
17. Pradhan M, Davidson B, Abeler V M, et al: DNA ploidy may be a prognostic marker in stage I and II serous adenocarcinoma of the endometrium. Virchows Archiv: an international journal of pathology 461:291-298, 2012
18. Levine D A, Network CGAR: Integrated genomic characterization of endometrial carcinoma. Nature 497:67, 2013
19. Goldman M, Craft B, Hastie M, et al: The UCSC Xena platform for public and private cancer genomics data visualization and interpretation. bioRxiv: 326470, 2019
20. Friedman J, Hastie T, Tibshirani R: Regularization paths for generalized linear models via coordinate descent. Journal of statistical software 33:1, 2010
21. Simon N, Friedman J, Hastie T, et al: Regularization paths for Cox's proportional hazards model via coordinate descent. Journal of statistical software 39:1, 2011
22. Szklarczyk D, Gable A L, Lyon D, et al: STRING v11: protein-protein association networks with increased coverage, supporting functional discovery in genome-wide experimental datasets. Nucleic Acids Res 47: D607-d613, 2019
23. Hayes M P, Douglas W, Ellenson L H: Molecular alterations of EGFR and PIK3CA in uterine serous carcinoma. Gynecologic oncology 113:370-373, 2009
24. Kogan L, Octeau D, Amajoud Z, et al: Impact of lower uterine segment involvement in type II endometrial cancer and the unique mutational profile of serous tumors. Gynecologic oncology reports 24:43-47, 2018
25. Fader A N, Roque D M, Siegel E, et al: Randomized Phase II Trial of Carboplatin-Paclitaxel Versus Carboplatin-Paclitaxel-Trastuzumab in Uterine Serous Carcinomas That Overexpress Human Epidermal Growth Factor Receptor 2/neu. Journal of Clinical Oncology 36:2044-2051, 2018
26. Kuhn E, Bahadirli-Talbott A, Shih I-M: Frequent CCNE1 amplification in endometrial intraepithelial carcinoma and uterine serous carcinoma. Modern Pathology 27:1014, 2013
27. Babicky M L, Harper M M, Chakedis J, et al: MST1R kinase accelerates pancreatic cancer progression via effects on both epithelial cells and macrophages. Oncogene: 1, 2019
28. Moser C, Lang S A, Hackl C, et al: Oncogenic MST1R activity in pancreatic and gastric cancer represents a valid target of HSP90 inhibitors. Anticancer research 32:427-437, 2012
29. Catenacci D V T, Cervantes G, Yala S, et al: RON (MST1R) is a novel prognostic marker and therapeutic target for gastroesophageal adenocarcinoma. Cancer biology & therapy 12:9-46, 2011
30. Ye J, Zhang Y, Cai Z, et al: Increased expression of immediate early response gene 3 protein promotes aggressive progression and predicts poor prognosis in human bladder cancer. BMC Urology 18:82, 2018
31. Garcia M N, Grasso D, Lopez-Millan M B, et al: IER3 supports KRASG12D-dependent pancreatic cancer development by sustaining ERK1/2 phosphorylation. J Clin Invest 124:4709-22, 2014
32. Jin H, Suh D S, Kim T H, et al: IER3 is a crucial mediator of TAp73f3-induced apoptosis in cervical cancer and confers etoposide sensitivity. Sci Rep 5:8367, 2015
33. Ling C, Ishiai M, Ali A M, et al: FAAP100 is essential for activation of the Fanconi anemia-associated DNA damage response pathway. Embo j 26:2104-14, 2007
34. Fujiwaki R, Takahashi K, Kitao M: Decrease in tumor volume and histologic response to intraarterial neoadjuvant chemotherapy in patients with cervical and endometrial adenocarcinoma. Gynecologic oncology 65:258-264, 1997
35. Kigawa J, Kanamori Y, Ishihara H, et al: Response rate and cell-cycle changes due to intra-arterial infusion chemotherapy with cisplatin and bleomycin for locally recurrent uterine cervical cancer. American journal of clinical oncology 15:474-479, 1992
36. Veldman-Jones M H, Brant R, Rooney C, et al: Evaluating robustness and sensitivity of the NanoString technologies nCounter platform to enable multiplexed gene expression analysis of clinical samples. Cancer research, 2015

We claim:

1. A method for predicting the outcome of a subject's overall survival (OS) for uterine serous carcinoma (USC) comprising:
obtaining a gene expression levels from a tumor sample from the subject of the genes selected from the group consisting of CNOT1, C1orf106, ACRC, MEIS3, HGS, GALNTL2, C8orf4, GALNTL4, IBTK, WNT7B, PHLDA2, DENND2A, C1orf126, IER3, FLJ35776, MYEOV, BTBD16, S100A10, MC1R, GNAL, RBMS2, MST1R, IL1R2, KCNE4, COL18A1, CUBN, CHRNA10, TAL1, S100A6, MMP10, S100A11, GPR124, EIF2B2, WDR17, OBFC2A, HABP2, C10orf47, GRIA3, LOC728264, COL4A4, ATG16L2, TXK, C17orf70, GPR111, COL1A1, HS3ST2, RHOV, SLC6A13, DOK4, DKK1, FLJ23867, PADI1, LIPG, LY6H, ZNF69, C2CD4A, C11orf41, VIL1, C11orf9, AG2, ERBB2, IL6, C3orf66, OVGP1, SAA4, NCOA, NPAS2, ITGA10, SH2D3A, C12orf27, CLDN14, F3, PAPPA and subcombinations thereof;
optionally normalizing the expression level to the expression of a housekeeping gene;
calculating a score of the gene expression levels using elastic net regression, wherein each gene is weighted; and
wherein a score of less than 9 indicates a longer OS for the subject, compared to a USC patient with a score higher than 9.

2. The method of claim 1, wherein the housekeeping gene is selected from the group consisting of actin, GAPDH and ubiquitin.

3. A method of selecting a treatment for a subject with USC comprising:
classifying subjects with USC into poor response to treatment groups or good response to treatment groups using the method of claim 1, wherein patients with a score of less than 9 indicates the patient will have a good response to standard treatment and patients with a score above 9 will have a good response to treatment with standard treatment; and
b. treating the patients with a score of less than 9 with standard treatment selected from the group consisting of resection, chemotherapy, and radiation.

4. A method of prognosis or classification of a subject having USC, comprising
determining the score of a subject using the method of claim 1;
wherein the stage progression of USC is early stage (I & II) if the score is below 9 or the USC is advanced stage (III & IV) if the score is above 9.

5. The method of claim 4, wherein subjects with a score higher than 9 or an advanced stage classification correlates with poor prognosis and a 5-year OS of 0% to 11.6%.

6. The method of claim 4, wherein subjects with a score lower than 9 or an early stage classification correlates with intermediate prognosis and a 5-year OS of 45% to 82.7%.
